(19) United States
(12) Patent Application Publication
(10) Pub. No.: US 2024/0206892 A1
(43) Pub. Date: Jun. 27, 2024

(54) THROMBUS REMOVAL SYSTEMS AND ASSOCIATED METHODS

(71) Applicant: Shifamed Holdings, LLC, Campbell, CA (US)

(72) Inventors: Aadel AL-JADDA, San Carlos, CA (US); Tom SAUL, Portland, OR (US); Kevin MULLER, San Carlos, CA (US); Amr SALAHIEH, Saratoga, CA (US)

(21) Appl. No.: 18/568,681

(22) PCT Filed: Jun. 10, 2022
(86) PCT No.: PCT/US2022/033050

§ 371 Date: Dec. 8, 2023

Related U.S. Application Data
(60) Provisional application No. 63/209,264, filed on Jun. 10, 2021.

(51) Int. Cl. A61B 17/22; A61B 17/00; A61M 1/00

(52) U.S. Cl. CPC A61B 17/22; A61B 17/00234; A61M 1/87; A61B 2017/00331; A61B 2017/22079; A61B 2217/005; A61M 2210/12

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to (1) at least partially fragment the thrombus and (2) provide an optical path for the imaging element, and an aspiration mechanism fluidly coupled to an aspiration lumen and configured to aspirate the fragmented thrombus.

### CROSS REFERENCE TO RELATED APPLICATIONS

This application claims the benefit of priority to U.S. Application No. 63/209,264, filed Jun. 10, 2021, which is herein incorporated by reference in its entirety.

### RELATED APPLICATIONS

This application is related to International Application No. PCT/US2021/020915, filed Mar. 4, 2021, and U.S. Application No. 63/190,784, filed May 19, 2021, the disclosures of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the vascular anatomy of the lungs, may not be effective in removing thrombotic material, and/or may lack the ability to provide sensor data or other feedback to the clinician during the thrombectomy procedure.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2D illustrate plan views of various configurations of irrigation ports and fluid streams of a thrombus removal system according to embodiments of the present technology.

FIGS. 3A-3H illustrate an elevation view of various configurations of irrigation ports of a thrombus removal system according to embodiments of the present technology.

FIGS. 4A-4H illustrate an elevation view of various configurations of irrigation ports and fluid streams of a thrombus removal system according to embodiments of the present technology.

FIGS. 5A-5G illustrate various configurations of irrigation ports of a thrombus removal system according to embodiments of the present technology.

FIGS. 6A-6C illustrate various embodiments of a thrombus removal system including a saline source, an aspiration system, and one or more controls for controlling irrigation and/or aspiration of the system.

FIGS. 7A-7D illustrate various embodiments of a funnel of a thrombus removal device.

FIGS. 8A-8B illustrate one embodiment of steering an elongate catheter.

FIG. 9 illustrates various visualization features that can be incorporated into an elongate catheter device.

### SUMMARY OF THE DISCLOSURE

A method for removing a thrombus from a blood vessel of a patient is provided, the method comprising introducing an elongate catheter having an expandable funnel to a thrombus location in a blood vessel in a closed configuration; expanding the funnel from the closed configuration to an open configuration to at least partially occlude the blood vessel; drawing at least a section of the thrombus into the distal portion; directing fluid toward the thrombus from at least two different points to break up the thrombus; aspirating at least a portion of the thrombus into the elongate catheter; collapsing the funnel into the closed configuration; and removing the elongate catheter from the blood vessel.

In some embodiments, the drawing is by suction applied via an aspiration lumen of the elongate catheter.

In one example, the fluid has an average velocity of at least 5 meters/second (m/s).

In some embodiments, the funnel is sealed in the closed configuration to prevent ingress of blood or tissue in the closed configuration.

In other embodiments, expanding the funnel further comprises expanding distal struts of the funnel with one or more pull wires.

In some embodiments, expanding the funnel further comprises dilating the blood vessel.

In some examples, expanding the funnel further comprises allowing shape memory distal struts of the funnel to automatically expand.

A system for removing a thrombus from a blood vessel of a patient, the system comprising an elongated catheter device having-a distal portion configured to be positioned within the blood vessel of the patient, the distal portion comprising an inner wall forming an aspiration lumen, an outer wall, a fluid lumen formed in a space between the inner and outer walls, and an auxiliary lumen formed in a space between the inner and outer walls, a funnel distal to the distal portion, the funnel having a closed configuration in which the funnel forms a tapered and sealed distal end, and an open configuration in which the funnel is configured to engage with a vessel wall and open the funnel to fluid communication with the blood vessel, at least two fluid ports adapted for fluid communication with the fluid lumen and configured to direct respective fluid streams into the distal portion; a proximal portion configured to be positioned external to the patient, the aspiration lumen and auxiliary lumen extending from the distal portion to the proximal portion; an aspiration mechanism positioned external to the patient and fluidly coupled with the aspiration lumen, the aspiration mechanism configured to reduce a pressure at the funnel (a) to engage the thrombus therewith and/or (b) to draw the thrombus and/or thrombus fragments proximally; and a fluid delivery mechanism configured to supply fluid through the fluid lumen.

In some embodiments, the funnel includes a plurality of proximal spines and a plurality of distal spines, wherein the plurality of distal spines are configured to be actuated to cause the funnel to change from the closed configuration to the open configuration and vis versa.

In other examples, the proximal spines are rotationally coupled to the distal spines.

In some embodiments, the distal spines are configured to be actuated with one or more pull wires.

In one embodiment, the system further comprises a sealing element disposed around some or all of a perimeter of the funnel, the sealing element being configured to prevent ingress of blood or tissue into the funnel when the funnel is in the closed configuration.

In some embodiments, comprising one or more visual aids disposed on or within the funnel to aid in visualization of the funnel during a procedure.

In some examples, the visual aids are selected from the group consisting of fiducial markers embedded in the funnel, fluoroscopic dyes injected in or around the funnel, gas chambers within the funnel, and balloons configured to be inflated to create echogenic regions under real-time imaging.

A method for removing a thrombus from a blood vessel of a patient is provided, the method comprising: introducing a funnel of an elongate catheter to a thrombus location in a blood vessel, wherein the funnel is in a closed configuration in which the funnel is tapered and scaled; expanding the funnel of the distal portion from the closed configuration to an open configuration to at least partially occlude the blood vessel; drawing at least a section of the thrombus into the distal portion; directing fluid toward the thrombus from at least two different points to break up the thrombus; and aspirating at least a portion of the thrombus into the elongate catheter.

In some embodiments, the drawing is by suction applied via an aspiration lumen of the elongate catheter.

In some embodiments, the fluid has an average velocity of at least 5 meters/second (m/s).

In some examples, the funnel is sealed to prevent ingress of blood or tissue in the closed configuration.

In other embodiments, expanding the funnel further comprises expanding distal struts of the funnel with one or more pull wires.

In some examples, expanding the funnel further comprises dilating the blood vessel.

In some embodiments, expanding the funnel further comprises allowing shape memory distal struts of the funnel to automatically expand.

### DETAILED DESCRIPTION

This application is related to disclosure in International Application No. PCT/US2021/020915, filed Mar. 4, 2021, the disclosure of which is incorporated by reference herein for all purposes.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally.” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. The fluid delivery mechanism and aspiration mechanism can be any as are described in embodiments of the Appendix. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clot that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

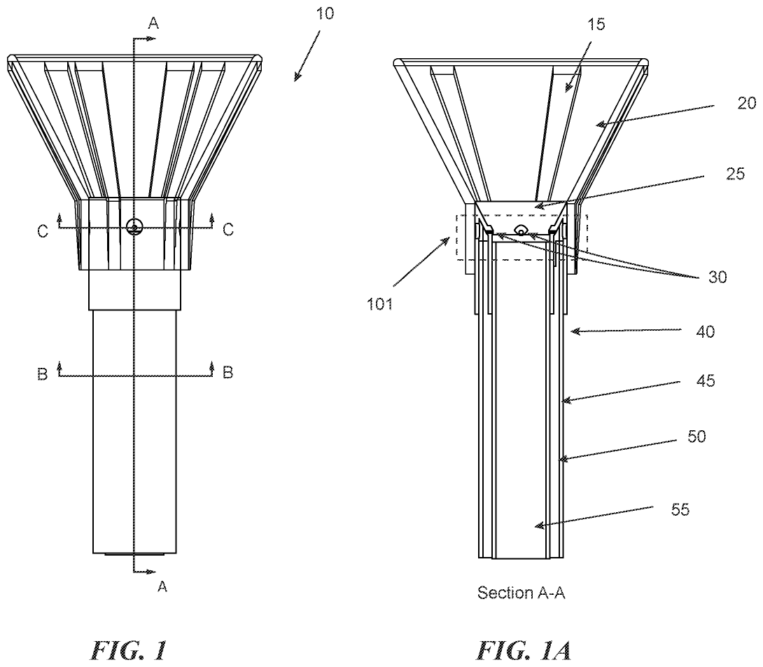

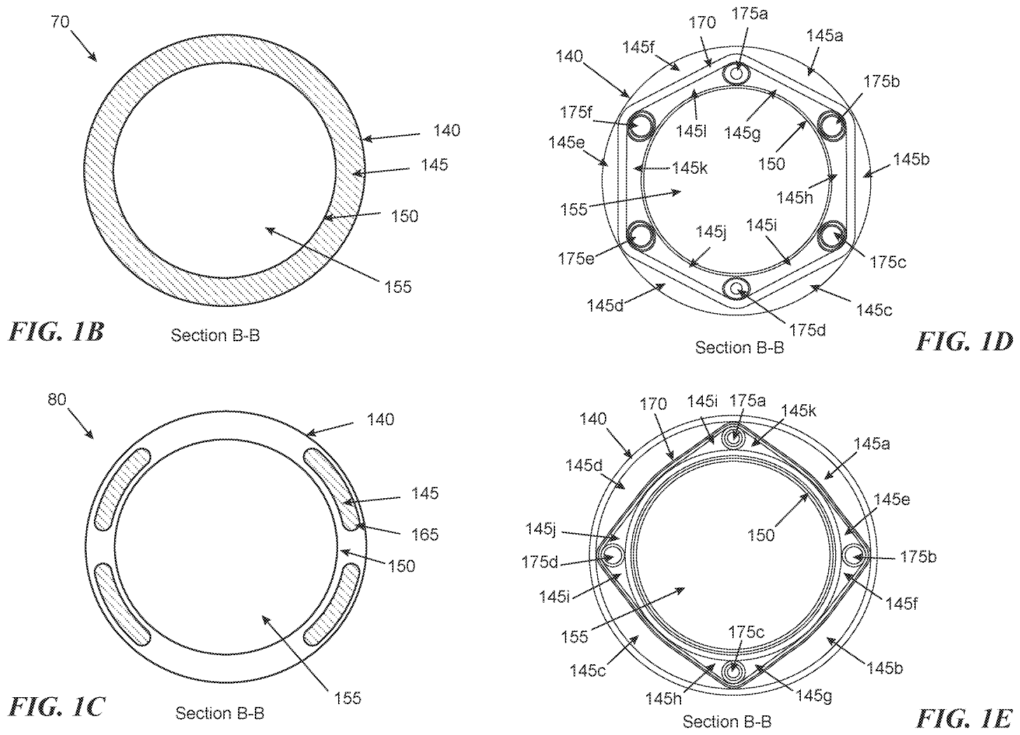

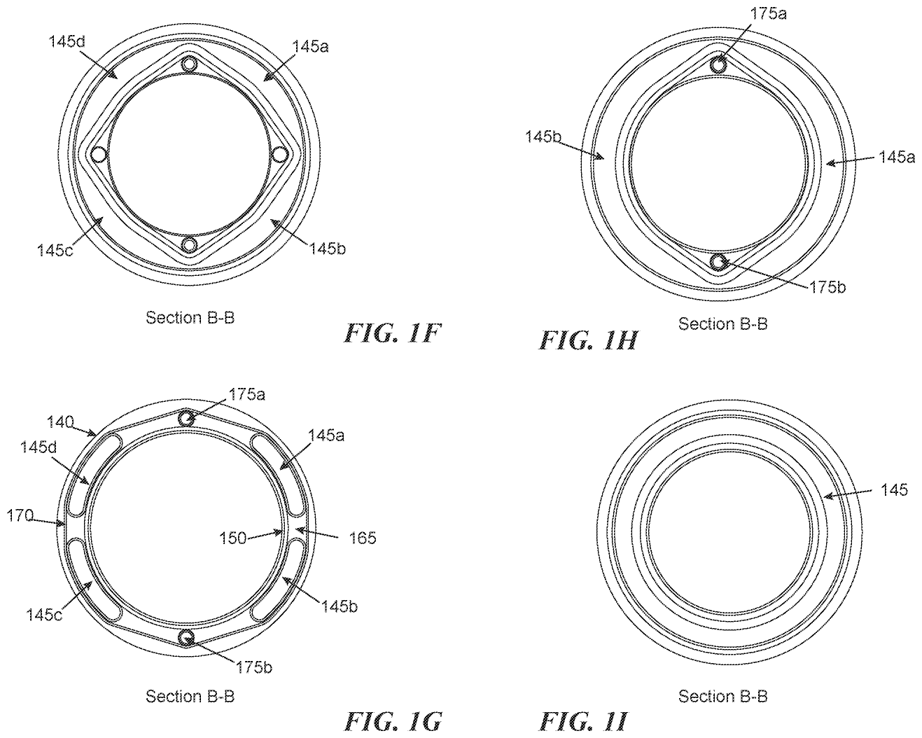

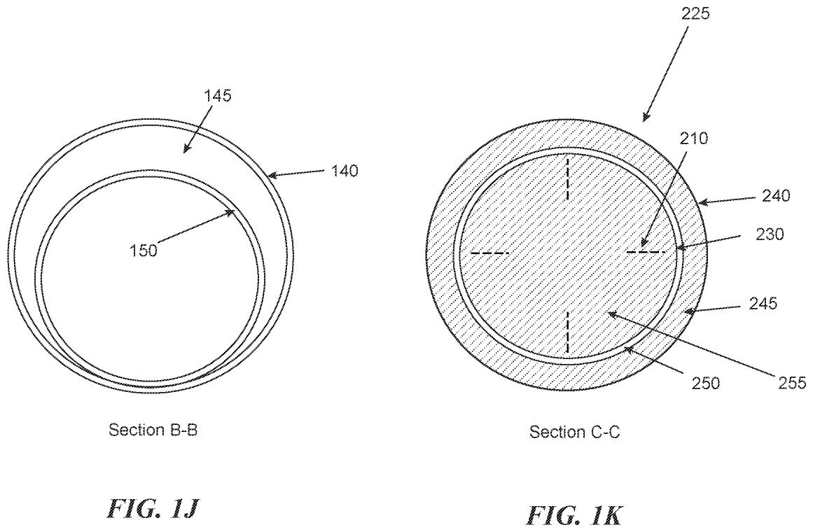

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The funnel can be formed according to any of the constructions described in the Appendix. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In general, the system can have an average flow velocity within the fluid lumen of at least 20 m/s to achieve consistent and successful aspiration of clots. In some embodiments, the jets or apertures are no smaller than 0.100″ to avoid undesirable spraying of fluid to the systems can have a minimum vacuum or aspiration pressure of 15 mmHg, to achieve the desired performance necessary to remove target clots.

The thrombus removal system can be sized and configured to access and remove thrombi in various locations or vessels within a patient's body. It should be understood that while the dimensions of the system may vary depending on the target location, generally the same features and components described herein will be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Referring to FIG. 1A, the funnel 20 can comprise a compliant material such as a polymer and include a plurality of grooves or slots 15 configured to receive one or more shape memory structures (not shown). In some embodiments, the shape memory structure can comprise a shape memory alloy such as nitinol. In some embodiments, the shape memory structures can be pre-biased to expand outwards, causing the funnel to expand when a constraining member, such as a delivery sheath, is removed from the funnel. In the illustrated example of FIG. 1A, the slots and shape memory structures extend generally along a longitudinal axis of the device (e.g., proximally to distally). In other embodiments, the slots and shape memory materials can be placed radially or in other configurations within the funnel.

Still referring to FIG. 1B, in one embodiment the funnel can extend over an outer wall of the device, as shown. This can ensure that the compliant material of the funnel covers or protects the patient from potentially sharp corners or edges of the outer wall or manifold of the thrombus removal device. In the illustrated embodiment, the funnel is positioned over the manifold, but in other embodiments, the manifold can be integrated with or positioned within the funnel. For example, in one embodiment the funnel can comprise a compliant cone-shaped bladder, and the manifold can be integrated into or positioned within the bladder. This can facilitate jets or apertures within the funnel itself, which can then receive fluid from the fluid lumens via the manifold.

The funnels described herein generally have a closed configuration that is useful for navigation and placement of the device, and an expanded configuration in which the funnel expands to contact and occlude the target vessel during therapy. In some embodiments, the funnel is passively or self-expanded (e.g., via a shape memory material) and in other embodiments the funnel is actively expanded (e.g., via a mechanical actuation). In additional embodiments, the funnel can be deployed via insertion or retraction of the two components of the device about one another. For example, an outer tube of the device could be attached to the funnel, and a concentric tube inside the outer tube could be the outer wall of the thrombus removal device. The outer tube could be moved relative to the inner tube to cause the funnel to expand or retract. In other embodiments, this relative motion to open and close could be done using rotation as well.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-141 and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The auxiliary ports can therefore be used to connect to the sensors, e.g., by electrical connection, optical connection, mechanical/wire connection, and/or fluid connection.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order. In embodiments where the jets are disposed on or within the funnel, the fluid lumens can be fluidly coupled to the funnel jets. Jets can be placed at any position within the funnel, such as along the edge of the funnel or in the main body of the funnel. Jets in the funnel can comprise a hole in the wall of the funnel, an extension of the fluid lumen that exits the funnel, or nozzles disposed in the funnel, etc. As mentioned above, this can be achieved by integrating or fluidly coupling the funnel with the manifold or directly to the fluid lumens. In some embodiments, the funnel may include a baffle structure or other systems for controlling fluids.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145c-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.50 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values. Generally, the length of the aperture or hole through the walls that is used to form the ports 230 needs to have a length sufficient to prevent formation of a spray or mist as the fluid exits the ports. Instead, a focused jet or stream is desired. Given the parameters described above, the length of the apertures through the walls that are used to form the ports should be at least 0.25 mm in length, optionally up to 0.4 mm or up to 1 mm or greater in length. Any lengths shorter than that may undesirably lead to mist or spray ejection from the ports, which will not effectively break up or macerate target clots.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

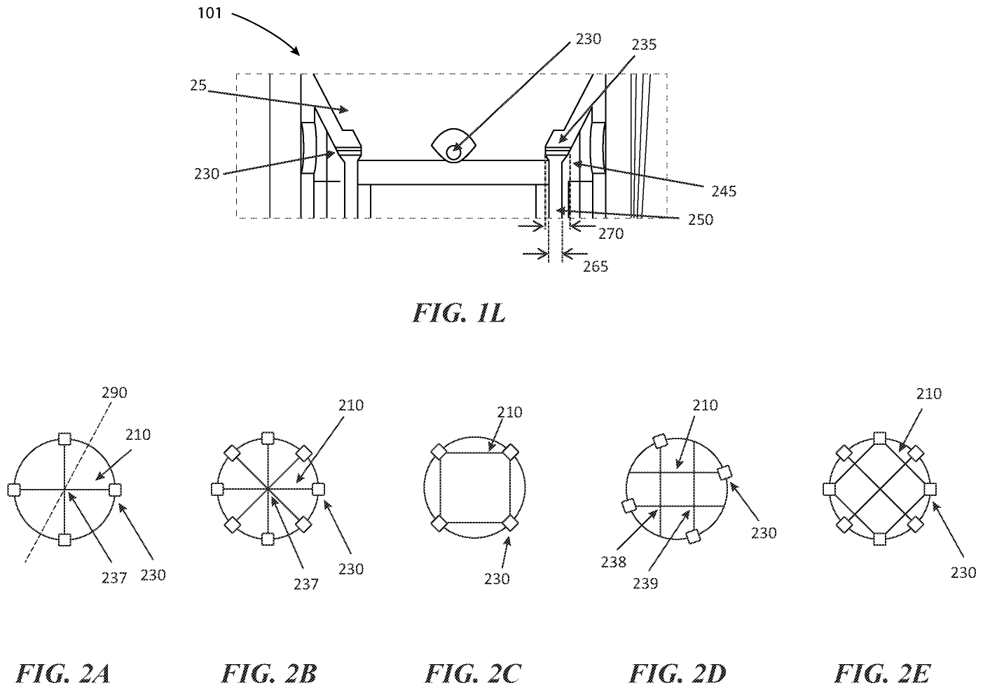

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which increase is with respect to individual fluid streams that are not directed to combine at the intersection. The increased fluid momentum and/or energy transfer at an intersection may advantageously fragment thrombus more efficiently and/or quickly. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. The targeted fluid velocity for fluid stream 210 can be any value within the range of aforementioned values. In some embodiments, at least two ports 230 are adapted to deliver respective fluid streams at different fluid velocities, for a given pressure of the fluid delivery mechanism. In some embodiments, at least two ports 230 are adapted to deliver respective fluid streams at the substantially the same fluid velocities, for a given pressure of the fluid delivery mechanism. In some embodiments, angular momentum is imparted to a thrombus by application of a) at least one fluid stream 210 that is directed at an oblique angle from a port 230, and/or b) at least two fluid streams 210 that have different fluid velocities. Advantageously, angular momentum produced in a thrombus may impart a (e.g., centrifugal) force that assists in fragmentation and removal of the thrombus. Advantageously, an increased cross-sectional area of the fluid lumen 145 reduces a required operating pressure of the fluid delivery mechanism to achieve a targeted fluid velocity of the fluid streams.

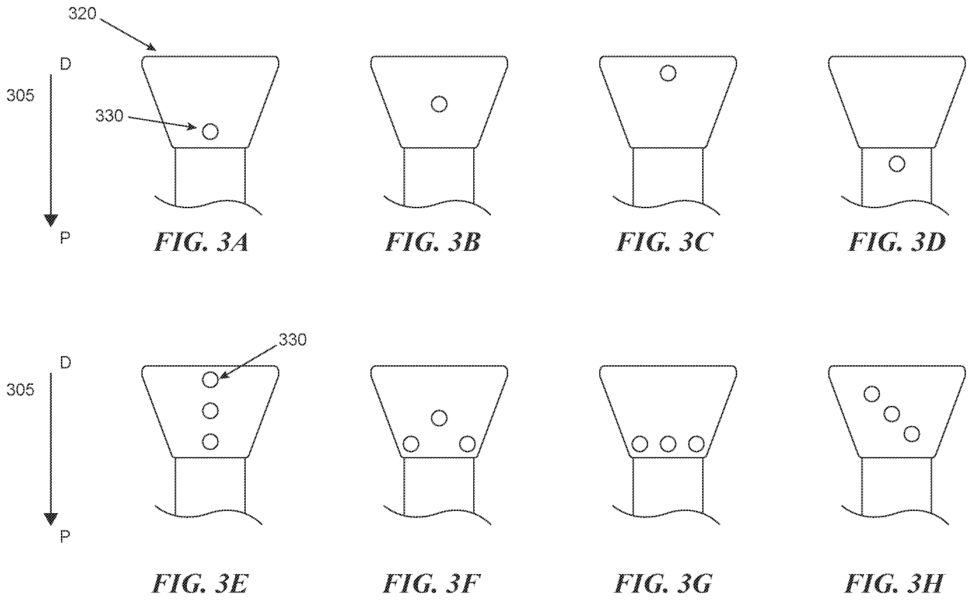

Referring to FIGS. 3A-3H, ports 330 can be arranged along various axial positions of the thrombus removal system. The thrombus removal system can include a flow axis 305 that is aligned with a general direction (e.g., distal-to-proximal) of flow for fluid that is aspirated therein. In some embodiments, a position of a port 330 comprises a) near a base of, b) in a middle portion of, c) in a distal portion of, or d) proximal to, a funnel portion 320 of the thrombus removal system. In some embodiments, at least two ports 330 are aligned along flow axis 305. In some embodiments, at least two ports 330 are arranged at a different axial positions along the flow axis 305. In some embodiments, at least two ports 330 are arranged (e.g., along a perimeter of the thrombus removal system) along a given axial position of the flow axis 305.

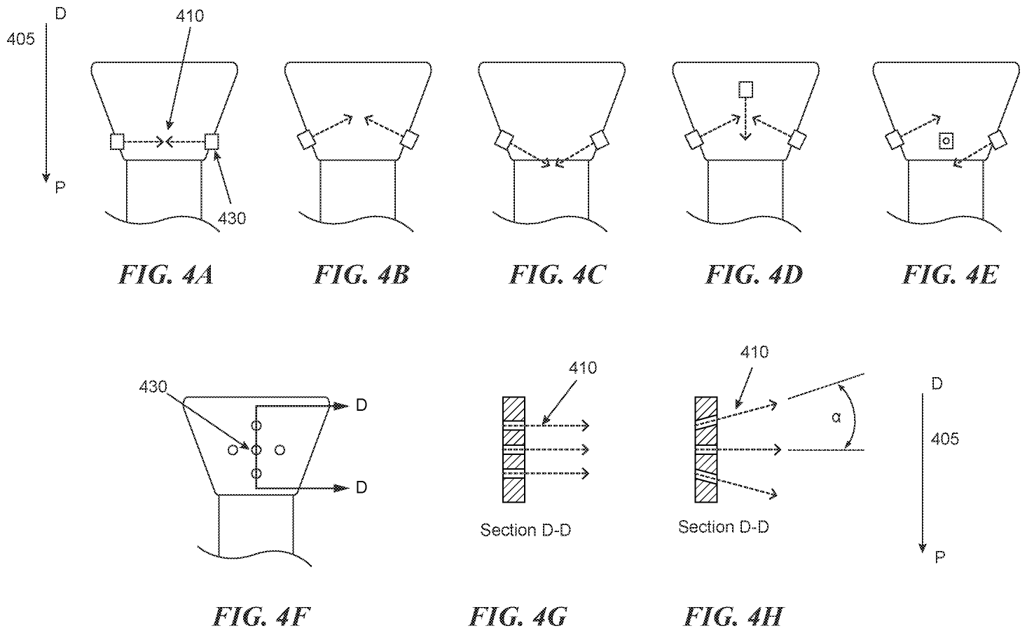

FIGS. 4A-4H depicts various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle α may characterize an angle α fluid stream 410 is directed with respect to an axis that is orthogonal to the flow axis 405 (e.g., as shown in section D-D of FIGS. 4G and 4H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

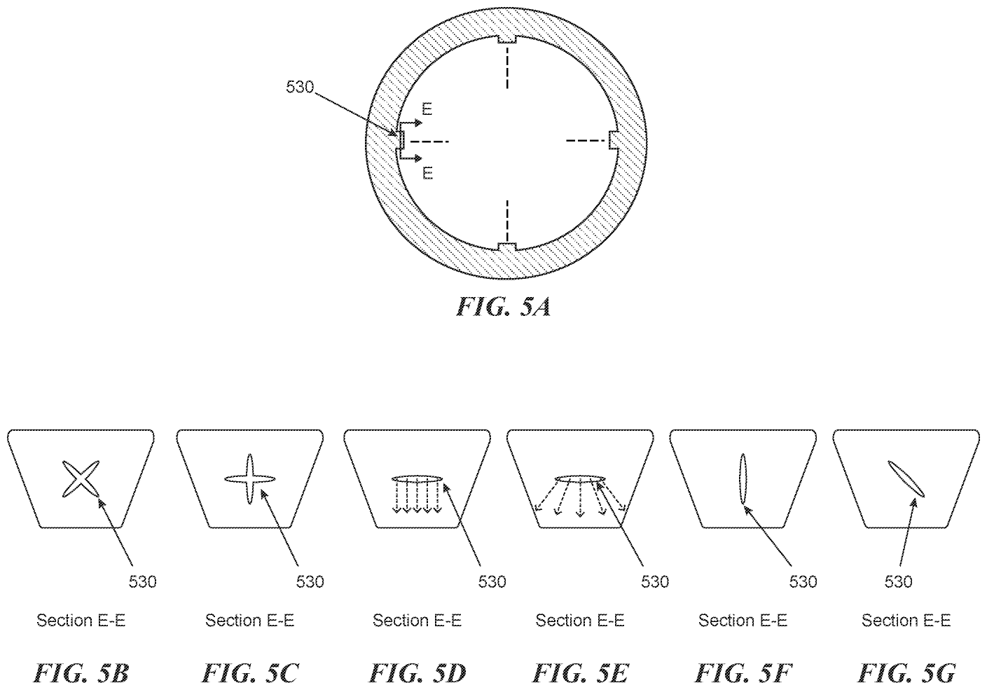

FIGS. 5A-5G illustrate a variety of exit aperture geometries with which ports 530 can be configured in accordance with embodiments of the present technology. Aperture geometries can comprise an oval, circular, cross (“x” shape), “t” shape, rectangle, or square shape. A fluid stream that is delivered from the port 530 can comprise substantially laminar flow (e.g., at the aperture), or a turbulent flow (e.g., that fans or outward).

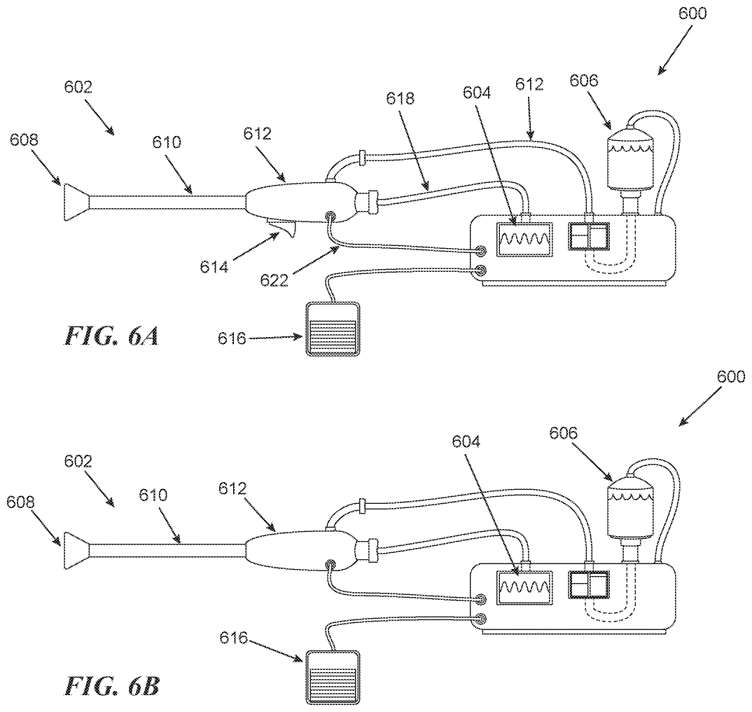

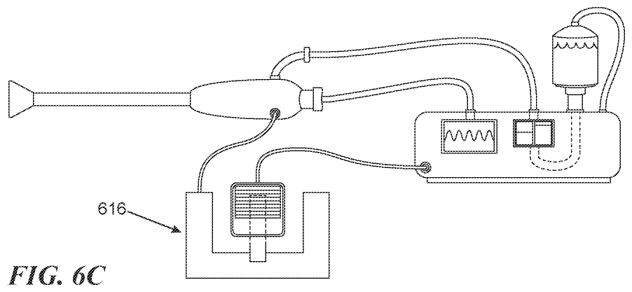

FIGS. 6A-6C illustrate various configurations of a thrombus removal system 600, including a thrombus removal device, 602, a vacuum source and cannister 604, and a fluid source 606. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 6A, the device can include a finger switch or trigger 614 and a foot pedal or switch 616. These can be used to control aspiration and irrigation, respectively. Alternatively, as shown in the embodiment of FIG. 6B, the device can include only a foot switch 614, which can be used to control both functions, or in FIG. 6C, the device can include only an overpedal 616, also used to control both functions. It is also contemplated that an embodiment could include only a finger switch to control both aspiration and irrigation functions. As shown in FIG. 6A, the vacuum source can be coupled to the aspiration lumen of the device with a vacuum line 618. Any clots or other debris removed from a patient during therapy can be stored in the vacuum cannister 604. Similarly, the fluid source (e.g., a saline bag) can be coupled to the fluid lumens of the device with a fluid line 620.

Still referring to FIG. 6A, electronics line 622 can couple any electronics/sensors etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both system in response to sensor data, such as pressure data, flow data, etc.

As is described above, aspiration occurs down the central lumen of the device and is provided by a vacuum pump in the console. The vacuum pump can include a container that collects any thrombus or debris removed from the patient.

### Additional Funnel Designs

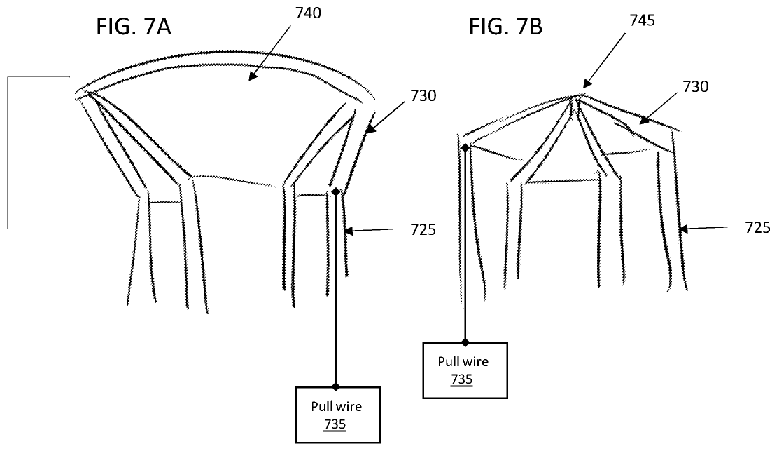

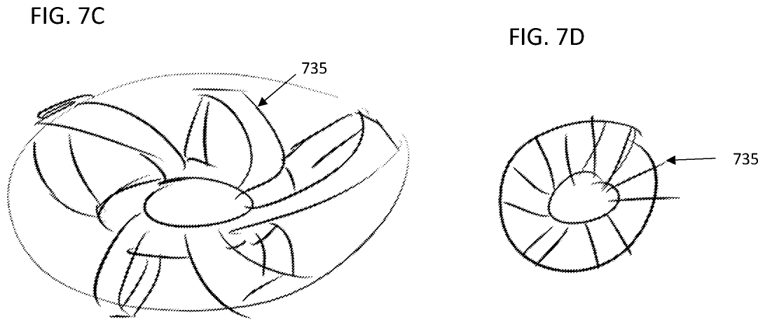

Referring to FIGS. 7A-7B, another embodiment of a funnel is shown that can include an open/expanded configuration (FIG. 7A) and a closed/contracted configuration (FIG. 7B). In the illustrated example, the funnel can include a plurality of proximal spines 725 and a plurality of distal spines 730. These spines can comprise a generally rigid material that can be integrated within or disposed within the compliant funnel. In some embodiments, the distal spines 730 can be mechanically actuated to transition between an open configuration and a closed configuration such as by one or more pull wires 735. In some examples, the proximal spines can be rotationally coupled to the distal spines (e.g., with a hinge or joint). In some embodiments, the pull wires can be disposed within auxiliary lumens in the shaft of the thrombus removal device (e.g., lumens 175a-175f in FIG. 1D).

In the open configuration, the distal spine 730 cause the funnel to expand, resulting in an opening 740 at a distal end of the funnel that can be configured to capture one or more clots into the funnel, aided by aspiration from the aspiration source. In some embodiments, the funnel in the open configuration can be actuated or expanded so as to dilate a target region such as a vessel. The rigidity provided by the distal spines in combination with the actuation forces provided by the pull wire(s) 735 can enable the device to provide a dilating or expansion force to targeted tissues or vessel walls.

The closed configuration forms a tapered distal end 745, tapered in the distal direction which can be used during delivery over a guidewire. In some embodiments, the tapered distal end is completely closed, sealed, or occluded when in the closed configuration, such that no blood or tissue can enter the funnel. For example, the perimeter of the funnel can include an o-ring, foam, skirt, or other sealing member configured to prevent ingress of fluids or tissues when in the closed configuration. The tapered distal end enables less traumatic delivery and allows the distal tip to more closely follow a guidewire during delivery. After placement of the device, the guidewire can be withdrawn, and the distal funnel can be actuated into the open configuration for use during therapy to occlude the target vessel and provide a larger entry zone for clot to be pulled into the aspiration lumen. In some embodiments, the funnel can be collapsed into the closed configuration to capture a thrombus in the closed funnel, and jets or aspiration can continue to operate to assist in clearing the thrombus from the funnel and from the device.

FIGS. 7C-7D illustrate two additional cross-sectional view of a funnel including one or more distal spines or supports 735. In FIG. 7C, the spines expand radially outward from a center of the aspiration lumen 55 and also include a slight curve or bend. In contrast, the distal spines 735 of FIG. 7D extend radially outward of the funnel in a straight line. Both of these designs can include shape memory spines that are pre-biased outwards to cause the funnel to expand when the thrombus removal device reaches the target tissue site.

### Steering and Navigation

As described above, the thrombus removal device can include steering mechanism(s) for navigating the device to a target treatment site or target thrombus. Steering can be performed in a variety of ways. As described above, one approach comprises one or more pull wires disposed in the auxiliary lumens as shown in FIGS. 1D, 1E, 1F, 1G, and 1H.

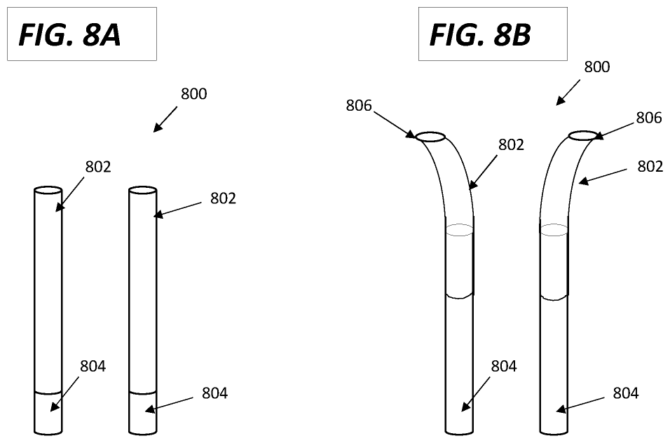

Alternatively, as shown in FIGS. 8A-8B, the system can incorporate a steering system 800 that includes two or more concentric tubes configured to be axially displaced relative to one another. FIG. 8A illustrates two pairs of centric tubes, each pair including a distal tube 802 and a proximal tube 804. The distal tube in each pair can have an inner diameter that is slightly wider than or equal to the outer diameter of the proximal tube, allowing the distal tube to concentrically slide over the proximal tube. In this embodiment, the distal tube can have a section that includes a preferential bend direction (e.g., shape memory), and the proximal tube can be substantially straight. In FIG. 8A, the distal tube pulled back proximally over the proximal tube so that it substantially covers the proximal tube. In this embodiment, the section of the distal tube with the preferential bend direction is retracted over the proximal tube, such that both tubes achieve a substantially straight configuration.

In FIG. 8B, the distal tube is advanced distally over the proximal tube, causing the section 806 of the distal tube with the preferential bend direction to assume its preferred state and bend in the preferential bend direction. As shown, in this embodiment, the left most pair of tubes can assume a preferential bend direction in a first direction (e.g., left in the frame) that is different than the preferential bend direction of the right most pair of tubes (e.g., right in the frame). In some embodiments, the tubes can be axially moved with respect to another with pull wires, push tubes, or other known mechanisms). The pairs of distal tubes can be inserted into auxiliary lumens of the thrombus removal device. During use, a first pair of tube can be used to steer the device in a first direction, and the second set of tubes can be used to steer the device in a second direction. The distal tube can be proximally moved over the proximal tube when no steering is desired. In other embodiments, only a single set of tubes is required with a single preferential bend direction. In this embodiment, the device itself can be rotated within the lumen to allow the single pair of tubes to be used for steering and guidance to a target location.

In this embodiment, wherein one or more of the tubes can include a section having a preferential bend direction. Additionally, the device could be steered using rotation wherein one or both devices have a preset bend.

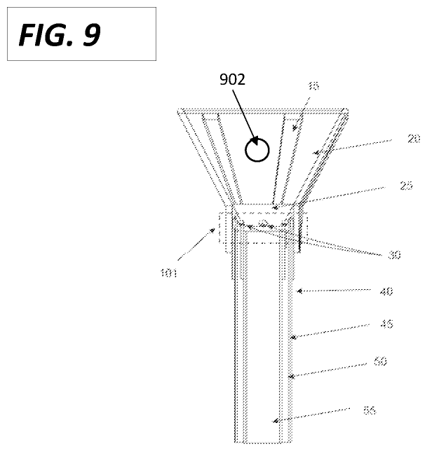

Referring to FIG. 9, to assist in steering and navigation, the systems and devices herein (such as the device of FIG. 1A) can further incorporate visual aids 902 to allow real-time visualization of the thrombus removal device during a procedure. These visual aids can include fiducial markers embedded in the device including in the funnel, fluoroscopic dyes injected in or around the funnel, the device, or the shaft, or features that result in echogenic regions within our around the device, including gas chambers within the funnel or device or small balloons that can be inflated to create echogenic regions under real-time imaging.

In some embodiments, the funnel may be used in conjunction with a dilator. In various embodiments, the dilator may be integrated into the funnel.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### Examples

Several aspects of the present technology are set forth in the following examples:

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising.” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A method for removing a thrombus from a blood vessel of a patient, the method comprising:
introducing an elongate catheter having an expandable funnel to a thrombus location in a blood vessel in a closed configuration;
expanding the funnel from the closed configuration to an open configuration to at least partially occlude the blood vessel;
drawing at least a section of the thrombus into the distal portion;
directing fluid toward the thrombus from at least two different points to break up the thrombus;
aspirating at least a portion of the thrombus into the elongate catheter;
collapsing the funnel into the closed configuration; and
removing the elongate catheter from the blood vessel.

2. The method of claim 1, wherein the drawing is by suction applied via an aspiration lumen of the elongate catheter.

3. The method of claim 1, wherein the fluid has an average velocity of at least 5 meters/second (m/s).

4. The method of claim 1, wherein the funnel is sealed in the closed configuration to prevent ingress of blood or tissue in the closed configuration.

5. The method of claim 1, wherein expanding the funnel further comprises expanding distal struts of the funnel with one or more pull wires.

6. The method of claim 1, wherein expanding the funnel further comprises dilating the blood vessel.

7. The method of claim 1, wherein expanding the funnel further comprises allowing shape memory distal struts of the funnel to automatically expand.

8. A system for removing a thrombus from a blood vessel of a patient, the system comprising:
an elongated catheter device having-
a distal portion configured to be positioned within the blood vessel of the patient, the distal portion comprising an inner wall forming an aspiration lumen, an outer wall, a fluid lumen formed in a space between the inner and outer walls, and an auxiliary lumen formed in a space between the inner and outer walls,
a funnel distal to the distal portion, the funnel having a closed configuration in which the funnel forms a tapered and sealed distal end, and an open configuration in which the funnel is configured to engage with a vessel wall and open the funnel to fluid communication with the blood vessel,
at least two fluid ports adapted for fluid communication with the fluid lumen and configured to direct respective fluid streams into the distal portion;
a proximal portion configured to be positioned external to the patient, the aspiration lumen and auxiliary lumen extending from the distal portion to the proximal portion;
an aspiration mechanism positioned external to the patient and fluidly coupled with the aspiration lumen, the aspiration mechanism configured to reduce a pressure at the funnel (a) to engage the thrombus therewith and/or (b) to draw the thrombus and/or thrombus fragments proximally; and
a fluid delivery mechanism configured to supply fluid through the fluid lumen.

9. The system of claim 8, wherein the funnel includes a plurality of proximal spines and a plurality of distal spines, wherein the plurality of distal spines are configured to be actuated to cause the funnel to change from the closed configuration to the open configuration and vis versa.

10. The system of claim 9, wherein the proximal spines are rotationally coupled to the distal spines.

11. The system of claim 9, wherein the distal spines are configured to be actuated with one or more pull wires.

12. The system of claim 8, further comprising a sealing element disposed around some or all of a perimeter of the funnel, the sealing element being configured to prevent ingress of blood or tissue into the funnel when the funnel is in the closed configuration.

13. The system of claim 8, further comprising one or more visual aids disposed on or within the funnel to aid in visualization of the funnel during a procedure.

14. The system of claim 13, wherein the visual aids are selected from the group consisting of fiducial markers embedded in the funnel, fluoroscopic dyes injected in or around the funnel, gas chambers within the funnel, and balloons configured to be inflated to create echogenic regions under real-time imaging.

15. A method for removing a thrombus from a blood vessel of a patient, the method comprising:
introducing a funnel of an elongate catheter to a thrombus location in a blood vessel, wherein the funnel is in a closed configuration in which the funnel is tapered and sealed;
expanding the funnel of the distal portion from the closed configuration to an open configuration to at least partially occlude the blood vessel;
drawing at least a section of the thrombus into the distal portion;
directing fluid toward the thrombus from at least two different points to break up the thrombus; and
aspirating at least a portion of the thrombus into the elongate catheter.

16. The method of claim 15, wherein the drawing is by suction applied via an aspiration lumen of the elongate catheter.

17. The method of claim 15, wherein the fluid has an average velocity of at least 5 meters/second (m/s).

18. The method of claim 15, wherein the funnel is sealed to prevent ingress of blood or tissue in the closed configuration.

19. The method of claim 15, wherein expanding the funnel further comprises expanding distal struts of the funnel with one or more pull wires.

20. The method of claim 15, wherein expanding the funnel further comprises dilating the blood vessel.

21. The method of claim 15, wherein expanding the funnel further comprises allowing shape memory distal struts of the funnel to automatically expand.
